(19) United States
(12) Patent Application Publication
(10) Pub. No.: US 2008/0306287 A1
(43) Pub. Date: Dec. 11, 2008

(54) Process for Preparing Tetrahydropyran-4-Carboxylic Acid Compound

(75) Inventors: Shigeyoshi Nishino, Yamaguchi (JP); Kenji Hirotsu, Yamaguchi (JP); Hidetaka Shima, Yamaguchi (JP); Takashi Harada, Yamaguchi (JP); Taku Nakamura, Yamaguchi (JP); Keiji Iwamoto, Yamaguchi (JP)

Correspondence Address: BIRCH STEWART KOLASCH & BIRCH, PO BOX 747, FALLS CHURCH, VA, 22040-0747, US

(73) Assignee: UBE INDUSTRIES, LTD., Ube-shi, Yamaguchi (JP)

(21) Appl. No.: 11/668,000

(22) PCT Filed: Nov. 18, 2005
(86) PCT No.: PCT/JP2005/021230

§ 371 Date: Jun. 11, 2008

(30) Foreign Application Priority Data
Nov. 18, 2004 (JP) 2004-334313
Nov. 26, 2004 (JP) 2004-341822
Jun. 2, 2005 (JP) 2005-162336

(51) Int. Cl. C07D 309/08

(52) U.S. Cl. 549/425

(57) ABSTRACT

The present invention is to provide a process for preparing tetrahydropyran-4-carboxylic acid represented by the formula (1):

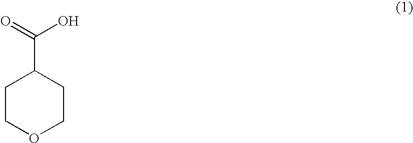

which comprises hydrolyzing a 4-cyanotetrahydropyran-4-carboxylic acid compound represented by the formula (2):

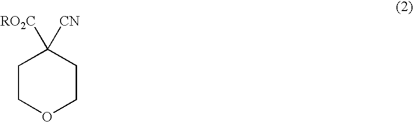

wherein R represents a hydrogen atom or a hydrocarbon group,
   in the presence of an acid; a process for preparing a tetrahydropyran-4-carboxylic acid ester which comprises reacting the tetrahydropyran-4-carboxylic acid represented by the above-mentioned formula (1) with an alcohol in the presence of an acid; and a process for preparing tetrahydropyran-4-carboxylic acid amide which comprises reacting the tetrahydropyran-4-carboxylic acid represented by the above-mentioned formula (1) with a halogenating agent to prepare tetrahydropyran-4-carboxylic acid halide, and then, reacting an amine compound to the resulting compound.

### TECHNICAL FIELD

The present invention relates to a process for preparing tetrahydropyran-4-carboxylic acid, tetrahydropyran-4-carboxylic acid ester, tetrahydropyran-4-carboxylic acid halide or tetrahydropyran-4-carboxylic acid amide from 4-cyanotetrahydropyran-4-carboxylic acid compound under mild conditions and simple and easy method. The tetrahydropyran-4-carboxylic acid compound is a compound useful as a starting material or a synthetic intermediate for a medicine, an agricultural chemical, etc.

### BACKGROUND ART

Heretofore, as a method for preparing tetrahydropyran-4-carboxylic acid, it has been known a method, for example, in which tetrahydropyran-4,4-dicarboxylic acid is heated to 185° C. to obtain tetrahydropyran-4-carboxylic acid with an isolation yield 64% (for example, see Patent literature 1). However, according to the above-mentioned method, there are problems that a high reaction temperature is required, and yet, yield is low, whereby it can not be satisfied as an industrial preparation method of tetrahydropyran-4-carboxylic acid.

Also, as a method for preparing tetrahydropyran-4-carboxylic acid ester, it has been known a method in which tetrahydropyran-4,4-dicarboxylic acid ester is subjected to decarboxylation (for example, see Patent literature 2). However, in this method, there are problems that a large amount of tetra-n-butyl phosphonium bromide is required, a reaction temperature is high, and yield of the desired product is low, and the like, so that it is not advantageous as an industrial process for preparing tetrahydropyran-4-carboxylic acid ester.

Moreover, as a method for preparing tetrahydropyran-4-carboxylic acid amide, there is disclosed a method for preparing tetrahydropyran-4-carboxylic acid amide, for example, by reacting tetrahydropyran-4-carboxylic acid with thionyl chloride to prepare tetrahydropyran-4-carboxylic acid chloride, and then, reacting the same with ammonia (for example, Non-patent literature 1). However, according to this method, there is no description about the details of the preparation method or yield, etc., so that it cannot be used as a reference for an industrial preparation method of a tetrahydropyran-4-carbonamide compound.

Patent literature 1: JP 2000-281672 A
Patent literature 2: WO 03/106418 A1 pamphlet
Non-patent literature 1: J. Chem. Soc., 1930, 2525.

### DISCLOSURE OF THE INVENTION

### Problems to be Solved by the Invention

An object of the present invention is to solve the above-mentioned problems, and to provide an industrially suitable process for preparing a tetrahydropyran-4-carboxylic acid compound which can prepare a tetrahydropyran-4-carboxylic acid, a tetrahydropyran-4-carboxylic acid ester, a tetrahydropyran-4-carboxylic acid halide or a tetrahydropyran-4-carboxylic acid amide from a 4-cyanotetrahydropyran-4-carboxylic acid compound with high yields under mild conditions with simple and easy method.

### Means to Solve the Problems

The present invention relates to a process for preparing tetrahydropyran-4-carboxylic acid represented by the formula (1):

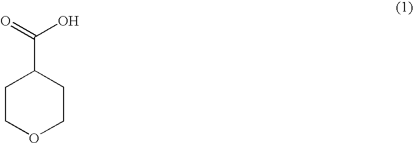

which comprises subjecting a 4-cyanotetrahydropyran-4-carboxylic acid compound represented by the formula (2):

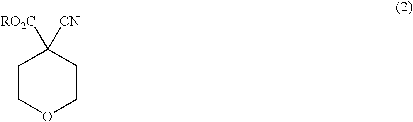

wherein R represents a hydrogen atom or a hydrocarbon group,
   to hydrolysis in the presence of an acid.

The present invention also relates to a process for preparing tetrahydropyran-4-carboxylic acid ester represented by the formula (4):

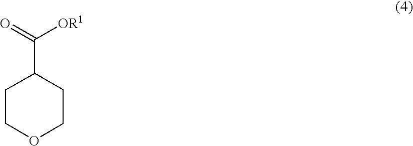

wherein R¹ represents a hydrocarbon group,
   which comprises reacting the tetrahydropyran-4-carboxylic acid represented by the above-mentioned formula (1) with an alcohol represented by the formula (3):

R¹OH (3)
wherein R¹ has the same meaning as defined above, in the presence of an acid.

The present invention further relates to a process for preparing tetrahydropyran-4-carboxylic acid halide represented by the formula (5):

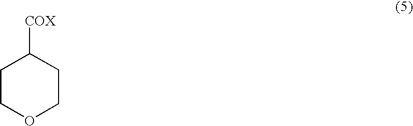

wherein X represents a halogen atom,
   which comprises reacting the tetrahydropyran-4-carboxylic acid represented by the above-mentioned formula (1) with a halogenating agent.

The present invention also relates to a process for preparing a tetrahydropyran-4-carboxylic acid amide compound represented by the formula (6):

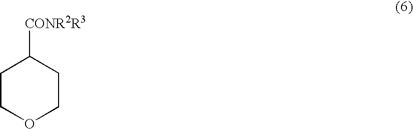

wherein R² and R³ each represents a hydrogen atom, a hydrocarbon group, an alkoxyl group, an aryloxyl group or an amino group; provided that the case where both of R² and R³ are amino groups is excluded; incidentally, R² and R³ may form a ring by binding to each other, and the ring may contain at least one hetero atom selected from an oxygen atom, a nitrogen atom and a sulfur atom,
   which comprises reacting the tetrahydropyran-4-carboxylic acid halide represented by the above-mentioned formula (2) with an amine compound represented by the formula (7):

R²R³NH (7)
wherein R² and R³ have the same meanings as defined above.

### BEST MODE FOR CARRYING OUT THE INVENTION

The 4-cyanotetrahydropyran-4-carboxylic acid compound to be used in the hydrolysis reaction of a preparation method of a tetrahydropyran-4-carboxylic acid according to the present invention is represented by the above-mentioned formula (2). In the formula (2), R represents a hydrogen atom or a hydrocarbon group, and as the hydrocarbon group, there may be mentioned, for example, an alkyl group having 1 to 6 carbon atoms such as a methyl group, ethyl group, propyl group, butyl group, pentyl group, hexyl group, etc.; an aralkyl group such as benzyl group, phenethyl group, phenylpropyl group, etc.; an aryl group such as a phenyl group, tolyl group, xylyl group, ethylphenyl group, etc., preferably an alkyl group having 1 to 4 carbon atoms, more preferably a methyl group and an ethyl group. Incidentally, these groups contain various kinds of isomers.

As the acid to be used in the hydrolysis reaction of the present invention, there may be mentioned, for example, an inorganic acid such as sulfuric acid, hydrochloric acid, nitric acid, phosphoric acid, hydrofluoric acid, hydrobromic acid, hydroiodic acid, etc.; a carboxylic acid such as formic acid, acetic acid, trifluoroacetid acid, trichloroacetic acid, etc.; a sulfonic acid such as methanesulfonic acid, benzenesulfonic acid, p-toluenesulfonic acid, etc., preferably used is sulfuric acid, hydrochloric acid, or phosphoric acid. Incidentally, these acids may be used alone or in admixture of two or more kinds.

An amount of the above-mentioned acid to be used is preferably 0.1 to 50 mols, more preferably 0.5 to 20 mols based on 1 mol of the 4-cyanotetrahydropyran-4-carboxylic acid compound.

As the solvent to be used in the hydrolysis reaction of the present invention, it is not specifically limited so long as it does not inhibit the reaction, and there may be mentioned, for example, water; an alcohol such as methanol, ethanol, isopropyl alcohol, t-butyl alcohol, etc.; an amide such as ,N-dimethylformamide, N,N-dimethylacetamide, N-methylpyrrolidone, etc.; a urea such as N,N′-dimethylimidazolidinone, etc.; a sulfoxide such as dimethyl sulfoxide, etc.; an ether such as diethyl ether, diisopropyl ether, tetrahydrofuran, dioxane, etc.; an aromatic hydrocarbon such as benzene, toluene, xylene, etc., preferably used is water or an alcohol. Incidentally, these solvents may be used alone or in admixture of two or more kinds.

An amount of the above-mentioned solvent to be used may be optionally adjusted depending on a degree of uniformity or condition of stirring of the reaction mixture, and preferably 0.01 to 100 ml, more preferably 0.1 to 20 ml based on 1 g of the 4-cyanotetrahydropyran-4-carboxylic acid compound.

In the above-mentioned reaction of the present invention, existence of water for hydrolysis reaction is essential, and as a source of water to be supplied, there may be mentioned water from an aqueous solution of an acid to be added as a catalyst, or water added as a solvent. An amount thereof is preferably 0.05 to 100 ml, more preferably 0.1 to 50 ml based on 1 g of the 4-cyanotetrahydropyran-4-carboxylic acid compound.

The hydrolysis reaction of the present invention can be carried out, for example, by a method in which the 4-cyanotetrahydropyran-4-carboxylic acid compound, the acid and a solvent are mixed, and reacted with stirring, and the like. A reaction temperature at this time is preferably 0 to 150° C., more preferably 20 to 120° C., and a reaction pressure is not particularly limited.

The tetrahydropyran-4-carboxylic acid can be obtained by the reaction of the present invention, and the compound can be isolated and purified by a general method, for example, after completion of the reaction, neutralization, extraction, filtration, concentration, distillation, recrystallization, crystallization, column chromatography, etc.

Next, a preparation method of the tetrahydropyran-4-carboxylic acid ester of the present invention is explained. The tetrahydropyran-4-carboxylic acid to be used in the reaction of the present invention is represented by the above-mentioned formula (1).

The alcohol to be used in the reaction of the present invention is represented by the above-mentioned formula (3). In the formula (3), R¹ is a hydrocarbon group, and there may be specifically mentioned, for example, an alkyl group having 1 to 6 carbon atoms such as a methyl group, ethyl group, propyl group, butyl group, pentyl group, hexyl group, etc.; an aralkyl group such as a benzyl group, phenethyl group, etc.; and an aryl group such as a phenyl group, tolyl group, etc., preferably an alkyl group having 1 to 4 carbon atoms, more preferably a methyl group and ethyl group. Incidentally, these groups contain various kinds of isomers.

An amount of the above-mentioned alcohol to be used is preferably 1 to 500 mols, more preferably 2 to 100 mols based on 1 mol of the tetrahydropyran-4-carboxylic acid.

The acid to be used in the reaction of the present invention may be mentioned, for example, an inorganic acid such as sulfuric acid, hydrofluoric acid, hydrochloric acid, hydrobromic acid, hydroiodic acid, nitric acid, phosphoric acid, etc.; a carboxylic acid such as formic acid, acetic acid, trifluoroacetic acid, trichloroacetic acid, etc.; a sulfonic acid such as methanesulfonic acid, benzenesulfonic acid, p-toluenesulfonic acid, etc., preferably used is sulfuric acid, hydrochloric acid or phosphoric acid. Incidentally, these acids may be used alone or in admixture of two or more kinds.

An amount of the above-mentioned acid to be used is preferably 0.01 to 10 mols, more preferably 0.05 to 5.0 mols based on 1 mol of the tetrahydropyran-4-carboxylic acid.

The reaction of the present invention is carried out in the presence or in the absence of a solvent. As the solvent to be used, it is not specifically limited so long as it does not inhibit the reaction, and there may be mentioned, for example, water; an aliphatic hydrocarbon such as hexane, heptane, cyclohexane, etc.; a carboxylic acid ester such as ethyl acetate, propyl acetate, butyl acetate, etc.; an ether such as diethyl ether, diisopropyl ether, dimethoxyethane, tetrahydrofuran, etc.; a halogenated aliphatic hydrocarbon such as methylene chloride, dichloromethane, etc.; an aromatic hydrocarbon such as toluene, xylene, etc.; an amide such as N,N-dimethylformamide, N,N-dimethylacetamide, N-methylpyrrolidone, etc.; a urea such as N,N′-dimethylimidazolidinone, etc.; a sulfoxide such as dimethyl sulfoxide, etc., preferably an ether, an aromatic hydrocarbon, an amide and a urea, more preferably tetrahydrofuran, toluene and N,N-dimethylacetamide are used. Incidentally, these solvents may be used alone or in admixture of two or more kinds.

An amount of the above-mentioned solvent to be used may be optionally adjusted depending on a degree of uniformity or condition of stirring of the reaction mixture, and preferably 0 to 100 ml, more preferably 0 to 50 ml based on 1 g of the tetrahydropyran-4-carboxylic acid.

The reaction of the present invention can be carried out, for example, by mixing tetrahydropyran-4-carboxylic acid, an alcohol and an acid, and reacting these materials with stirring, and the like. A reaction temperature at this time is preferably 20 to 150° C., more preferably 30 to 130° C., and a reaction pressure is not particularly limited.

Incidentally, the tetrahydropyran-4-carboxylic acid ester which is the final product can be isolated and purified, for example, after completion of the reaction, according to the conventional manner such as neutralization, extraction, filtration, concentration, distillation, recrystallization, column chromatography, etc.

Next, preparation methods of the tetrahydropyran-4-carboxylic acid halide and the tetrahydropyran-4-carboxylic acid amide of the present invention are explained. The preparation method of the present invention comprises two steps of a halogenation step and an amidation step as shown by the following reaction scheme (1):

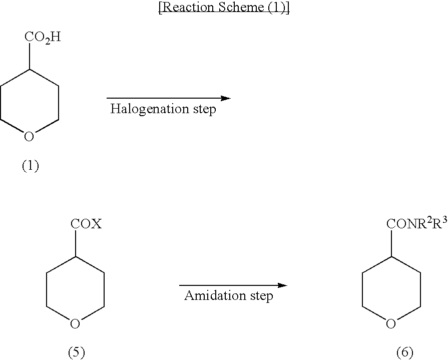

wherein X, R² and R³ has the same meaning as defined above.
   These two steps are sequentially explained.

### (1) Halogenation Step

As the halogenating agent to be used in the halogenation step of the present invention, there may be mentioned, for example, chlorine, bromine, thionyl chloride, thionyl bromide, oxalyl chloride, sulfuryl chloride, sulfuryl bromide, triphenylphosphine dichloride, triphenylphosphine dibromide, etc., preferably thionyl chloride and/or oxalyl chloride is/are used. Incidentally, these halogenated compounds may be used alone or in admixture of two or more kinds (limited to those having the same halogen atom).

An amount of the above-mentioned halogenating agent to be used is preferably 1.0 to 10 mols, more preferably 1.0 to 5.0 mols based on 1 mol of the tetrahydropyran-4-carboxylic acid.

The halogenation step of the present invention is desirably carried out in the presence of an organic solvent, and as the organic solvent to be used, it is not specifically limited so long as it does not inhibit the reaction, and there may be mentioned, for example, an aliphatic hydrocarbon such as hexane, heptane, octane, cyclohexane, etc.; an aromatic hydrocarbon such as benzene, toluene, xylene, etc.; an amide such as N,N-dimethylformamide, N,N-dimethylacetamide, N-methylpyrrolidone, etc.; a urea such as N,N′-dimethylimidazolidinone, etc.; a sulfoxide such as dimethyl sulfoxide, etc.; a nitrile such as acetonitrile, propionitrile, benzonitrile, etc.; an ether such as diethyl ether, diisopropyl ether, dimethoxyethyl ether, tetrahydrofuran, dioxane, etc.; a halogenated aliphatic hydrocarbon such as dichloromethane, chloroform, dichloroethane, etc.; a halogenated aromatic hydrocarbon such as chlorobenzene, dichlorobenzene, etc., preferably an aromatic hydrocarbon, an ether and/or an amide is/are used. Incidentally, these solvents may be used alone or in admixture of two or more kinds.

An amount of the above-mentioned solvent to be used may be optionally adjusted depending on a degree of uniformity or condition of stirring of the reaction mixture, and preferably 0.5 to 100 ml, more preferably 1.0 to 20 ml based on 1 g of the tetrahydropyran-4-carboxylic acid compound.

The halogenation step of the present invention can be carried out, for example, by the method in which the tetrahydropyran-4-carboxylic acid, the halogenating agent and an organic solvent are mixed and reacted under stirring, and the like. A reaction temperature at this time is preferably 0 to 150° C., more preferably 20 to 110° C., and a reaction pressure is not particularly limited.

The tetrahydropyran-4-carboxylic acid halide can be obtained according to the reaction of the present invention, and the material can be isolated and purified, after completion of the reaction, according to the conventional manner such as neutralization, extraction, filtration, concentration, distillation, recrystallization, crystallization, column chromatography, etc., and the next amidation step may be carried out without isolation and purification.

Incidentally, in the tetrahydropyran-4-carboxylic acid halide represented by the formula (5), X is a halogen atom, and there may be mentioned, for example, a fluorine atom, chlorine atom, bromine atom and iodine atom.

### (2) Amidation Step

The amine compound to be used in the amidation step of the present invention is represented by the above-mentioned formula (7). In the formula (7), R² and R³ each represent a hydrogen atom, a hydrocarbon group, an alkoxyl group or an aryloxyl group, and as the hydrocarbon group, there may be specifically mentioned, for example, an alkyl group having 1 to 10 carbon atoms such as a methyl group, ethyl group, propyl group, butyl group, pentyl group, hexyl group, heptyl group, octyl group, nonyl group, decyl group, etc.; a cycloalkyl group having 3 to 8 carbon atoms such as a cyclopropyl group, cyclobutyl group, cyclopentyl group, cyclohexyl group, cycloheptyl group, cyclooctyl group, etc.; an aralkyl group having 7 to 12 carbon atoms such as a benzyl group, phenethyl group, phenylpropyl group, etc.; an aryl group having 6 to 20 carbon atoms such as a phenyl group, p-tolyl group, naphthyl group, anthryl group, etc.; an amino group (provided that the case where both of R² and R³ are simultaneously amino groups is excluded). Also, as the alkoxyl group, there may be specifically mentioned, for example, an alkoxyl group having 1 to 6 carbon atoms such as a methoxyl group, ethoxyl group, propoxyl group, isopropoxyl group, butoxyl group, etc., an alkoxyl group-substituted alkoxyl group such as a methoxyethoxyl group, etc., and as the aryloxyl group, there may be specifically mentioned an aryloxyl group such as a phenoxyl group, benzyloxyl group, etc. Incidentally, these groups contain various kinds of isomers.

Incidentally, R² and R³ may form a ring by combining with each other, and the ring formed by binding may be specifically mentioned, for example, a pyrrolidine ring, piperidine ring, piperazine ring, morpholine ring, etc.

The above-mentioned hydrocarbon group, alkoxyl group or aryloxyl group may have a substituent(s). As the substituent(s), there may be mentioned a substituent(s) formed through a carbon atom, a substituent(s) formed through an oxygen atom, a substituent(s) formed through a nitrogen atom, a substituent(s) formed through a sulfur atom, etc.

As the above-mentioned substituent(s) formed through a carbon atom, there may be mentioned, for example, an alkyl group having 1 to 6 carbon atoms such as a methyl group, ethyl group, propyl group, butyl group, pentyl group, hexyl group, etc.; a cycloalkyl group having 3 to 10 carbon atoms such as a cyclopropyl group, cyclobutyl group, cyclopentyl group, cyclohexyl group, cycloheptyl group, etc.; an alkenyl group having 2 to 10 carbon atoms such as a vinyl group, allyl group, propenyl group, cyclopropenyl group, cyclobutenyl group, cyclopentenyl group, etc.; a heterocyclic group such as a quinolyl group, pyridyl group, pyrrolidyl group, pyrrolyl group, furyl group, thienyl group, etc.; an aryl group such as a phenyl group, tolyl group, fluorophenyl group, xylyl group, biphenylyl group, naphthyl group, anthryl group, phenanthryl group, etc.; an acyl group (which may be in a form of acetal) such as an acetyl group, propionyl group, acryloyl group, pivaloyl group, cyclohexylcarbonyl group, benzoyl group, naphthoyl group, toluoyl group, etc.; a carboxyl group; an alkoxycarbonyl group such as a methoxycarbonyl group, ethoxycarbonyl group, etc.; an aryloxycarbonyl group such as a phenoxycarbonyl group, etc.; a halogenated alkyl group such as a trifluoromethyl group, etc.; and a cyano group. Incidentally, these groups contain various kinds of isomers.

As the above-mentioned substituent(s) formed through an oxygen atom, there may be mentioned, for example, a hydroxyl group; an alkoxyl group such as a methoxyl group, ethoxyl group, propoxyl group, butoxyl group, pentyloxyl group, hexyloxyl group, heptyloxyl group, benzyloxyl group, etc.; an aryloxyl group such as a phenoxyl group, toluoyloxyl group, naphthyloxyl group, etc. Incidentally, these groups contain various kinds of isomers.

As the above-mentioned substituent(s) formed through a nitrogen atom, there may be mentioned, for example, an amino group; a primary amino group such as a methylamino group, ethylamino group, butylamino group, cyclohexylamino group, phenylamino group, naphthylamino group, etc.; a secondary amino group such as a dimethylamino group, diethylamino group, dibutylamino group, methylethylamino group, methylbutylamino group, diphenylamino group, N-methyl-N-methanesulfonylamino group, etc.; a heterocyclic amino group such as a morpholino group, piperidino group, piperazinyl group, pyrazolidinyl group, pyrrolidino group, indolyl group, etc.; and an, imino group. Incidentally, these groups contain various kinds of isomers.

As the above-mentioned substituent(s) formed through a sulfur atom, there may be mentioned, for example, a mercapto group; a thioalkoxyl group such as a thiomethoxyl group, thioethoxyl group, thiopropoxyl group, etc.; a thioaryloxyl group such as a thiophenoxyl group, thiotoluoyloxyl group, thionaphthyloxyl group, etc., and the like. Incidentally, these groups contain various kinds of isomers.

As the above-mentioned amine compound, there may be mentioned, for example, ammonia; a primary amine such as methylamine, ethylamine, n-propylamine, isopropylamine, n-butylamine, isobutylamine, sec-butylamine, tert-butylamine, etc.; a secondary amine such as methylethylamine, dimethylamine, diethylamine, di-n-propylamine, diisopropylamine, di-n-butylamine, diisobutylamine, di-sec-butylamine, ditert-butylamine, etc.; an alkoxylalkylamine such as methoxymethylamine, methoxyethylamine, ethoxymethylamine, ethoxyethylamine, etc.; an aralkylamine such as benzylamine, benzylmethylamine, etc.; an arylamine such as aniline, methylaniline, etc.; a heterocyclic amine such as morpholine, piperazine, etc.; hydrazine; a substituted hydrazine such as methylhydrazine, dimethylhydrazine, phenylhydrazine, etc. Of these, preferred are ammonia; a monoalkylamine such as methylamine, ethylamine, etc.; a dialkylamine such as dimethylamine, diethylamine, etc.; and an alkoxylalkylamine such as methoxymethylamine, etc., more preferably ammonia, diethylamine or methoxymethylamine.

An amount of the above-mentioned amine compound to be used is preferably 1.0 to 50 mole(s), more preferably 1.0 to 10 mole(s), particularly preferably 1.0 to 5.0 mole(s) based on 1 mole of tetrahydropyran-4-carboxylic acid halide. Incidentally, the amine compound to be used may be either of the form such as an aqueous solution or an acid salt (for example, hydrochloride, etc.), and the like. Also, when an acid salt of an amine compound is used, it may be used by neutralizing with a suitable base.

The amidation step of the present invention is desirably carried out in the presence of a solvent. As the solvent to be used, it is not specifically limited so long as it does not inhibit the reaction, and there may be mentioned, for example, water; an aliphatic hydrocarbon such as hexane, heptane, octane, cyclohexane, etc.; an aromatic hydrocarbon such as benzene, toluene, xylene, etc.; an amide such as N,N-dimethylformamide, N,N-dimethylacetamide, N-methylpyrrolidone, etc.; a urea such as N,N′-dimethylimidazolidinone, etc.; a sulfoxide such as dimethyl sulfoxide, etc.; a nitrile such as acetonitrile, propionitrile, benzonitrile, etc.; an ether such as diethyl ether, diisopropyl ether, dimethoxyethyl ether, tetrahydrofuran, dioxane, etc.; a halogenated aliphatic hydrocarbon such as chloroform, dichloroethane, etc.; and a halogenated aromatic hydrocarbon such as chlorobenzene, etc., preferably an aromatic hydrocarbon, an amide, a nitrile and an ether, more preferably toluene, N,N-dimethylformamide, acetonitrile, and/or tetrahydrofuran is/are used. Incidentally, these solvents may be used alone or in admixture of two or more kinds.

An amount of the above-mentioned solvent to be used may be optionally adjusted depending on a degree of uniformity or condition of stirring of the reaction mixture, and it is preferably 0.1 to 100 ml, more preferably 1.0 to 20 ml based on 1 g of the tetrahydropyran-4-carboxylic acid halide.

The amidation step of the present invention can be carried out, for example, by the method that the tetrahydropyran-4-carboxylic acid halide, the amine compound (or a salt thereof or an aqueous solution thereof) and a solvent are mixed, and they are reacted with stirring, and the like. A reaction temperature at this time is preferably −20 to 150° C., more preferably −10 to 110° C., and a reaction pressure is not particularly limited. Incidentally, when an acid salt of an amine compound is used as the amine compound, it is preferred to add a base (for example, triethylamine, etc.) to the reaction system.

The tetrahydropyran-4-carboxylic acid amide compound can be obtained according to the reaction of the present invention, and the material can be isolated and purified, after completion of the reaction, according to the conventional manner such as neutralization, extraction, filtration, concentration, distillation, recrystallization, crystallization, column chromatography, etc.

Uses of the compounds of the present invention obtained as mentioned above are described, for example, in WO 2003/3083954, pp. 47 to 48 as a use of pyrancarboxylic acid for a starting material and synthetic intermediate of a medicine, an agricultural chemical, etc., in WO 2005/032484, p. 278 as a use of pyrancarboxylic acid amide for a starting material and synthetic intermediate of a medicine, an agricultural chemical, etc., in WO 2001/087870, p. 22 as a use of pyrancarboxylic acid ester for a starting material and synthetic intermediate of a medicine, an agricultural chemical, etc., and in Japanese Laid-Open Patent Publication No. 2003-183254, p. 18 as a use of pyrancarboxylic acid halide for a starting material and synthetic intermediate of a medicine, an agricultural chemical, etc.

### EXAMPLES

Next, the present invention will be explained more specifically by referring to Examples, but the scope of the present invention is not limited by these.

### Example 1

### Synthesis of tetrahydropyran-4-carboxylic acid

In a vessel made of a glass, having an inner volume of 20 ml and equipped with a stirring device, a thermometer were charged 0.85 g (5.0 mmol) of methyl 4-cyanotetrahydropyran-4-carboxylate and 3.5 ml (21 mmol) of 6 mol/l hydrochloric acid, and the mixture was reacted at 100° C. for 7 hours under stirring. After completion of the reaction, when the reaction mixture was analyzed (Internal standard method by gas chromatography), 0.51 g (Reaction yield: 78%) of tetrahydropyran-4-carboxylic acid was formed.

### Example 2

### Synthesis of tetrahydropyran-4-carboxylic acid

In a vessel made of a glass, having an inner volume of 20 ml and equipped with a stirring device, a thermometer were charged 1.0 g (6.5 mmol) of 4-cyanotetrahydropyran-4-carboxylic acid and 10 ml (60 mmol) of 6 mol/l hydrochloric acid, and the mixture was reacted at 100° C. for 9 hours under stirring. After completion of the reaction, when the reaction mixture was analyzed (Internal standard method by gas chromatography), 0.74 g (Reaction yield: 88%) of tetrahydropyran-4-carboxylic acid was formed.

### Example 3

### Synthesis of tetrahydropyran-4-carboxylic acid

In a vessel made of a glass, having an inner volume of 20 ml and equipped with a stirring device, a thermometer were charged 0.5 g (3.2 mmol) of 4-cyanotetrahydropyran-4-carboxylic acid, 0.5 g (3.0 mmol) of methyl 4-cyanotetrahydropyran-4-carboxylate and 10 ml (60 mmol) of 6 mol/l hydrochloric acid, and the mixture was reacted at 100° C. for 9 hours under stirring. After completion of the reaction, when the reaction mixture was analyzed (Internal standard method by gas chromatography), 0.68 g (Reaction yield: 84%) of tetrahydropyran-4-carboxylic acid was formed.

### Example 4

### Synthesis of tetrahydropyran-4-carboxylic acid methyl

In a vessel made of a glass, having an inner volume of 100 ml and equipped with a stirring device, a thermometer and a dropping funnel were charged 3.0 g (23.1 mmol) of tetrahydropyran-4-carboxylic acid, 452 mg (4.6 mmol) of conc. sulfuric acid and 50 ml of methanol, and the mixture was reacted at 60 to 70° C. for 5 hours. After completion of the reaction, the reaction mixture was concentrated, to the concentrate were added 50 ml of ethyl acetate and 10 ml of a saturated aqueous sodium chloride solution, and the organic layer was collected by separation. Then, the organic layer was washed with 10 ml of saturated brine, and dried over anhydrous magnesium sulfate. After filtration, the filtrate was concentrated under reduced pressure to obtain 3.17 g (Isolation yield: 95%) of methyl tetrahydropyran-4-carboxylate as pale yellowish liquid.

Physical properties of the methyl tetrahydropyran-4-carboxylate were as follows.

CI-MS (m/e); 145 (M+1)

¹H-NMR (CDCl₃, δ (ppm)); 1.79 to 1.88 (4H, m), 2.50 to 2.60 (1H, m), 3.42-3.47 (2H, m), 3.70 (3H, s), 3.93 to 3.99 (2H, m)

### Example 5

### Synthesis of tetrahydropyran-4-carboxylic acid chloride

In a vessel made of a glass, having an inner volume of 50 ml and equipped with a stirring device, a thermometer and a reflux condenser were charged 6.85 g (52.6 mmol) of tetrahydropyran-4-carboxylic acid, 9.79 g (82.3 mmol) of thionyl chloride and 10 ml of toluene, and the mixture was reacted at 80° C. for 1.5 hours under stirring. After completion of the reaction, the reaction mixture was concentrated under reduced pressure to obtain 7.81 g (Isolation yield; 100%) of tetrahydropyran-4-carboxylic acid chloride as pale brownish liquid.

Physical properties of the tetrahydropyran-4-carboxylic acid chloride were as follows.

¹H-NMR (CDCl₃, δ (ppm)); 1.79 to 1.93 (2H, m), 1.99 to 2.06 (2H, m), 2.91 to 3.00 (1H, m), 3.40 to 3.49 (2H, m), 3.97 to 4.03 (2H, m)

CI-MS (m/e); 131 (M+1)

### Example 6

### Synthesis of tetrahydropyran-4-carboxylic acid amide

In a vessel made of a glass, having an inner volume of 100 ml and equipped with a stirring device, a thermometer and a reflux condenser were charged 6.30 g (38.5 mmol) of tetrahydropyran-4-carboxylic acid chloride synthesized in the same method as in Example 5 and 20 g (329 mmol) of 28% by weight aqueous ammonia, and the mixture was reacted at 0° C. for 6 hours under stirring. After completion of the reaction, the reaction mixture was filtered, and the obtained filtrate was dried to obtain 4.84 g (Isolation yield; 62%) of tetrahydropyran-4-carboxylic acid amide was white crystals.

Physical properties of the tetrahydropyran-4-carboxylic acid amide were as follows.

¹H-NMR (CDCl₃, δ (ppm)); 1.46 to 1.62 (4H, m), 2.26 to 2.52 (1H, m), 3.28 to 3.34 (2H, m), 3.81 to 3.87 (2H, m), 6.77 to 7.24 (2H, d)

CI-MS (m/e); 130 (M+1)

### Example 7

### Synthesis of tetrahydropyran-4-carboxylic acid diethylamide

In a vessel made of a glass, having an inner volume of 50 ml and equipped with a stirring device, a thermometer and a reflux condenser were charged 1.00 g (6.73 mmol) of tetrahydropyran-4-carboxylic acid chloride synthesized in the same method as in Example 5, 1.08 g (14.8 mmol) of diethylamine and 5 ml of toluene, and the mixture was reacted at 20 to 30° C. for 1 hour under stirring. After completion of the reaction, 10 ml of water was added to the reaction mixture, and the organic layer was separated. Then, the aqueous layer was extracted with 20 ml of ethyl acetate three times, and the extracts and the organic layer were combined and dried over magnesium sulfate. After filtration, the filtrate was concentrated under reduced pressure, and the concentrate was purified by silica gel column chromatography (Eluent; ethyl acetate) to obtain 0.83 g (Isolation yield; 66%) of tetrahydropyran-4-carboxylic acid diethylamide as pale yellowish liquid.

Physical properties of the tetrahydropyran-4-carboxylic acid diethylamide were as follows.

¹H-NMR (CDCl₃, δ (ppm)); 1.08 to 1.24 (6H, m), 1.54 to 1.60 (2H, m), 1.93 to 1.99 (2H, m), 2.63 to 2.67 (1H, m), 3.30 to 3.86 (4H, m), 3.41 to 3.49 (2H, m), 4.00 to 4.06 (2H, m)

CI-MS (m/e); 186 (M+1)

### Example 8

### Synthesis of tetrahydropyran-4-carboxylic acid methoxymethylamide

In a vessel made of a glass, having an inner volume of 100 ml and equipped with a stirring device, a thermometer, a dropping funnel and a reflux condenser were charged 3.94 g (40.3 mmol) of methoxymethylamine hydrochloride and 40 ml of acetonitrile, and while maintaining the liquid temperature to 0° C., 8.16 g (80.6 mmol) of triethylamine was gradually added to the mixture. Then, 5.00 g (33.6 mmol) of tetrahydropyran-4-carboxylic acid chloride having purity of 86% synthesized in the same manner as in Example 5 was gradually added to the mixture and the resulting mixture was reacted at 0° C. for 1.5 hours under stirring. After completion of the reaction, the reaction mixture was concentrated under reduced pressure, 30 ml of toluene and 20 ml of water were added to the concentrate and the organic layer was separated, and the aqueous layer was extracted with 30 ml of toluene twice. The organic layer and the extracts were combined, and dried over anhydrous magnesium sulfate. After filtration, the filtrate was concentrated under reduced pressure to obtain 5.81 g (isolation yield; 100%) of tetrahydropyran-4-carboxylic acid methoxymethylamide as pale yellowish liquid.

Physical properties of the tetrahydropyran-4-carboxylic acid methoxymethylamide were as follows.

¹H-NMR (CDCl₃, δ (ppm)); 1.80 to 1.93 (2H, m), 1.99 to 2.05 (2H, m), 2.91 to 2.98 (1H, m), 3.40 to 3.49 (2H, m), 3.96 to 4.03 (2H, m)

CI-MS (m/e); 174 (M+1)

### UTILIZABILITY IN INDUSTRY

The present invention relates to a process for preparing a tetrahydropyran-4-carboxylic acid compound from a 4-cyanotetrahydropyran-4-carboxylic acid compound under mild conditions and a simple and easy method, a novel process for preparing a tetrahydropyran-4-carboxylic acid ester and a process for preparing a tetrahydropyran-4-carboxylic acid amide compound from the tetrahydropyran-4-carboxylic acid. The tetrahydropyran-4-carboxylic acid, tetrahydropyran-4-carboxylic acid ester and tetrahydropyran-4-carboxylic acid amide compound are useful compounds for a starting material or a synthetic intermediate of a medicine, an agricultural chemical, etc.

1. A process for preparing tetrahydropyran-4-carboxylic acid represented by the formula (1): 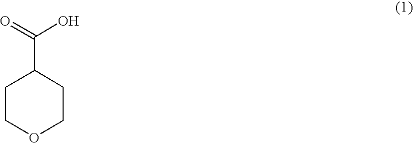
which comprises hydrolyzing a 4-cyanotetrahydropyran-4-carboxylic acid compound represented by the formula (2): 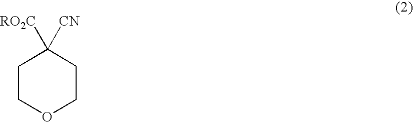
wherein R represents a hydrogen atom or a hydrocarbon group,
in the presence of an acid.

2. The process for preparing the tetrahydropyran-4-carboxylic acid according to claim 1, wherein the reaction is carried out in a solvent.

3. A process for preparing a tetrahydropyran-4-carboxylic acid ester represented by the formula (4): 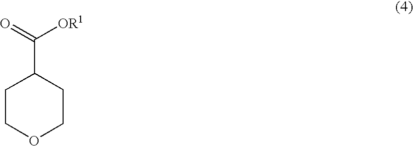
wherein R¹ represents a hydrocarbon group,
which comprises reacting tetrahydropyran-4-carboxylic acid represented by the formula (1): 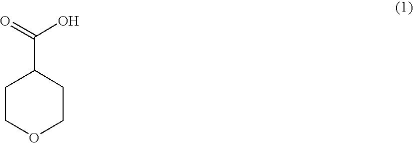
with an alcohol represented by the formula (3):
R¹OH (3)
wherein R¹ has the same meaning as defined above, in the presence of an acid.

4. The process for preparing the tetrahydropyran-4-carboxylic acid ester according to claim 3, wherein the tetrahydropyran-4-carboxylic acid represented by the formula (1) is obtained by hydrolyzing a 4-cyanotetrahydropyran-4-carboxylic acid compound represented by the formula (2): 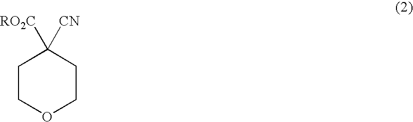
wherein R represents a hydrogen atom or a hydrocarbon group.

5. A process for preparing a tetrahydropyran-4-carboxylic acid halide represented by the formula (5): 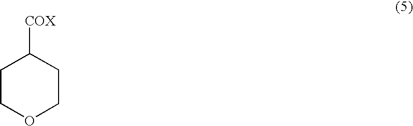
wherein X represents a halogen atom,
which comprises reacting tetrahydropyran-4-carboxylic acid represented by the formula (1): 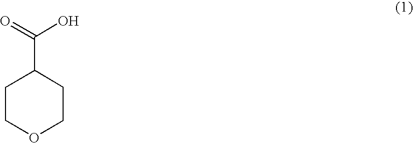
with a halogenating agent.

6. The process for preparing the tetrahydropyran-4-carboxylic acid halide according to claim 5, wherein the tetrahydropyran-4-carboxylic acid represented by the formula (1) is obtained by hydrolyzing a 4-cyanotetrahydropyran-4-carboxylic acid compound represented by the formula (2): 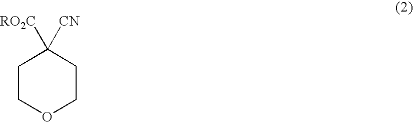
wherein R represents a hydrogen atom or a hydrocarbon group.

7. A process for preparing a tetrahydropyran-4-carboxylic acid amide compound represented by the formula (6): 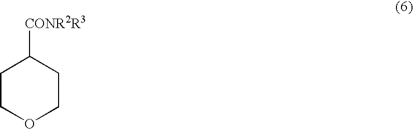
wherein R² and R³ each represent a hydrogen atom, a hydrocarbon group, an alkoxyl group, an aryloxyl group or an amino group; provided that the case where both of R² and R³ are amino groups is excluded;
incidentally, R² and R³ may form a ring by binding to each other, and the ring may contain at least one hetero atom selected from an oxygen atom, a nitrogen atom and a sulfur atom,
which comprises reacting a tetrahydropyran-4-carboxylic acid halide represented by the formula (5): 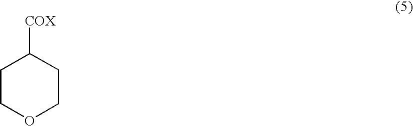
wherein X has the same meaning as defined above, with an amine compound represented by the formula (7):
R²R³NH (7)
wherein R² and R³ has the same meaning as defined above.

8. The process for preparing a tetrahydropyran-4-carboxylic acid amide compound according to claim 7, wherein the tetrahydropyran-4-carboxylic acid halide represented by the formula (5) is obtained by reacting tetrahydropyran-4-carboxylic acid represented by the formula (1): 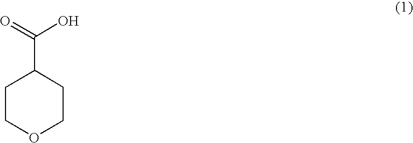
with a halogenating agent.

9. The process for preparing a tetrahydropyran-4-carboxylic acid amide compound according to claim 8, wherein the tetrahydropyran-4-carboxylic acid represented by the formula (1) is obtained by hydrolyzing a 4-cyanotetrahydropyran-4-carboxylic acid compound represented by the formula (2): 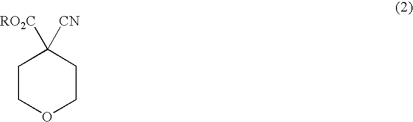
wherein R represents a hydrogen atom or a hydrocarbon group.

10. The process for preparing a tetrahydropyran-4-carboxylic acid amide compound according to claim 7, wherein the amine compound is at least one selected from the group consisting of ammonia, methylamine, ethylamine, dimethylamine, diethylamine and methoxymethylamine.
